(19) United States
(12) Patent Application Publication
(10) Pub. No.: US 2008/0138449 A1
(43) Pub. Date: Jun. 12, 2008

(54) COMPOSITION AND METHOD FOR SUPPORTING THERMOGENESIS AND LIPID OXIDATION

(75) Inventors: Marvin A. Heuer, Mississauga (CA); Shan Chaudhuri, Brampton (CA); Ken Clement, Mississauga (CA)

Correspondence Address: TORYS LLP, 79 WELLINGTON ST. WEST, SUITE 3000, TORONTO, ON, M5K 1N2, omitted

(73) Assignee: H3 FORMULATIONS LTD., Mississauga (CA)

(21) Appl. No.: 11/945,518

(22) Filed: Nov. 27, 2007

Related U.S. Application Data
(60) Provisional application No. 60/868,847, filed on Dec. 6, 2006.

(51) Int. Cl. A61K 36/754; A61K 36/82; A61P 3/00

(52) U.S. Cl. 424/729; 424/769

(57) ABSTRACT

A nutritional composition and method is provided for promoting weight loss or weight maintenance in an individual by supporting and enhancing thermogenesis and lipid oxidation comprising therapeutically effective amounts of yohimbine or derivatives thereof, an effective amount of Evodia rutaecarpia extract and a source of an effective amount of L-carnitine or derivatives thereof.

### CROSS REFERENCE TO RELATED APPLICATION

This application claims the benefit of U.S. Provisional Patent Application Ser. No. 60/868,847, filed Dec. 6, 2006, the content of which is incorporated by reference.

### FIELD OF THE INVENTION

The present invention relates to a nutritional composition for supporting weight loss or weight maintenance in an individual.

### BACKGROUND OF THE INVENTION

One of the main contributing factors in obesity is overeating, which results in an excess of energy being consumed in relation to the amount of energy being expended. The excess energy is then stored largely as fat. A simplified determination of an individual's body weight is essentially governed by the net effect of energy consumed versus energy expended. Daily energy expenditure consists of three components: basal metabolic rate, adaptive thermogenesis and physical activity (Westerterp K R. Diet induced thermogenesis. Nutr Metab (Lond). 2004 Aug. 18; 1(1):5).

Adaptive thermogenesis can be defined as the regulated production of heat in response to environmental changes in temperature and diet (Joosen A M, Westerterp K R. Energy expenditure during overfeeding. Nutr Metab (Lond). 2006 Jul. 12; 3:25). This is related to the observation that the weight gain resulting from overeating does not typically equal what would be predicted based on the amount of excess calories (Welle S, Campbell R G. Stimulation of thermogenesis by carbohydrate overfeeding. Evidence against sympathetic nervous system mediation. J Clin Invest. 1983 April; 71(4):916-25).

Three macronutrients constitute the food that we consume: protein, carbohydrate and fat. The term lipid is typically used synonymously with fat; however fat is a specific type of lipid wherein it is a solid at room temperature (Molecular Biology of the Cell, 3^{rd} Edition. 1994. Bruce Alberts, Dennis Bray, Julian Lewis, Martin Raff, Keith Roberts, and James D. Watson. Garland Publishing, pg 658-659).

Use of food for energy is accomplished by oxidation. Adenosine triphosphate (ATP) is generated through the process of oxidative phosphorylation within mitochondria. Through this process, the transfer of electrons to oxygen is coupled to the transfer of protons across the mitochondrial membranes to maintain a gradient which is used to drive the formation of ATP by the enzyme ATP synthase. Increase energy demand i.e. increased requirement for ATP, is normally linked to increased oxidative phosphorylation where protons transported into the mitochondria are used to generate ATP which is then exported in exchange for ADP (adenosine diphosphate). Agents are known that cause ‘uncoupling’ of electron transport from ATP synthesis allowing the dissipation of energy as heat i.e. thermogenesis (Molecular Biology of the Cell, 3^{rd} Edition. 1994. Bruce Alberts, Dennis Bray, Julian Lewis, Martin Raff, Keith Roberts, and James D. Watson. Garland Publishing, pg 682). The uncoupling proteins (UCP) are a group of transporter proteins in the mitochondrial membrane which dissipates the proton gradient generated through oxidative phosphorlyation, thereby facilitating thermogenesis (Ledesma A, de Lacoba M G, Rial E. The mitochondrial uncoupling proteins. Genome Biol. 2002; 3(12):REVIEWS3015). Therefore, agents that act as uncoupling agents affect the activity of UCPs. Adrenergic signaling has been shown to regulate UCPs (Kozak U C, Held W, Kreutter D, Kozak L P. Adrenergic regulation of the mitochondrial uncoupling protein gene in brown fat tumor cells. Mol Endocrinol. 1992 May; 6(5):763-72). The specific effects of adrenergic signaling depends on a number of factors including the adrenergic receptor subtype expressed on the target cell (Lafontan M, Berlan M. Fat cell adrenergic receptors and the control of white and brown fat cell function. J Lipid Res. 1993 July; 34(7):1057-91) however, it can also signal an a increase in thermogenesis and reduction in fat (Lafontan M, Berlan M. Fat cell alpha 2-adrenoceptors: the regulation of fat cell function and lipolysis. Endocr Rev. 1995 December; 16(6):716-38).

In order for fat to be oxidized it must first be transported to the mitochondria. Before fat can be transported in to the mitochondria it must be activated to facilitate transport via specific transporters. A key component of this transport is carnitine. Fat is bound by ester formation to carnitine for transport into the mitochondria by the a series of enzymatic reactions (Eaton S, Bartlett K, Pourfarzam M. Mammalian mitochondrial beta-oxidation. Biochem J. 1996 Dec. 1; 320 (Pt 2):345-57).

While ingestion of protein and carbohydrate has been shown to stimulate protein and carbohydrate oxidation, the ingestion of fat does not stimulate oxidation of fat (Schutz Y, Flatt J P, Jéquier E. Failure of dietary fat intake to promote fat oxidation: a factor favouring the development of obesity. Am J Clin Nutr. 1989 50:307-314). Furthermore, the oxidation of fat has been shown to be inhibited by the ingestion of carbohydrates (Jéquier E. Carbohydrates as a source of energy. Am J Clin Nutr. 1994 59(Suppl):682S-685S).

Therefore, it is advantageous to an individual concerned with reducing or maintaining body weight or body fat to promote thermogenesis and thus encourage the oxidation of fat.

### SUMMARY OF THE INVENTION

The foregoing needs and other needs and objectives that will become apparent for the following description are achieved in the present invention, which comprises a nutritional composition and method directed at promoting weight loss in an individual by supporting increased thermogenesis and fat oxidation. The composition comprises a source of an effective amount of Yohimbine or derivatives thereof, an effective amount of Evodia rutaecarpia extract and a source of an effective amount of L-carnitine or derivatives thereof.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional supplement and method for promoting weight loss in an individual by enhancing fat oxidation and encouraging thermogenesis.

The term ‘fat’ as used herein is understood to represent a form of lipid and includes related forms such as triglycerides, cholesterol, high density lipoproteins (HDL), low density lipoproteins (LDL) and fatty acids.

The term ‘mobilization of fat’ as used herein is understood to mean the release of fat from storage into the blood stream for use as energy in cells (Molecular Biology of the Cell, 3^{rd} Edition. 1994. Bruce Alberts, Dennis Bray, Julian Lewis, Martin Raff, Keith Roberts, and James D. Watson. Garland Publishing, pg 658).

It is herein understood that increases or enhancements of thermogenesis include all mechanisms and modes of increasing energy expenditure in an individual at the cost of energy that would otherwise be considered excess energy, destined to be stored as fat or glycogen, thus adding to body weight.

Furthermore, it is also herein understood that increases or enhancements of lipid oxidation include, but are not limited to the promotion of any or all of the following: the breakdown of stored or circulating fat, the transport of fat to a cell, the transport of fat into a cell, the transport of fat to the mitochondria, the transport of fat into the mitochondria and the sequential oxidation of fat within the mitochondria. It is further understood that a number of biochemical steps involving numerous enzymes and mechanisms are involved in the process of lipid oxidation and enhancement of any specific process may improve the oxidation of lipids.

In a preferred embodiment of the present invention, the composition is comprised of a source of an effective amount of Yohimbine or derivatives thereof, Evodia rutaecarpia extract and a source of an effective amount of L-carnitine or derivatives thereof, for supporting or enhancing thermogenesis and fat oxidation.

In another embodiment of the present invention, the composition is further comprised of one or more of the following: Yerba Mate, Black tea, Theobroma cacao, White tea dry leaf extract, Caffeine Anhydrous and White willow Bark, for support or enhancement of thermogenesis and fat oxidation.

Yohimbine

Yohimbe from the inner bark of the Corynanthe yohimbe tree has been used traditionally as an aphrodisiac. Yohimbe is a source of the indole alkaloid yohimbine, which in humans is metabolized to yield two hydroxylated forms: 10- and 11-hydroxyyohimbine (Le Verge R, Le Corre P, Chevanne F, Doe De Maindreville M, Royer D, Levy J. Determination of yohimbine and its two hydroxylated metabolites in humans by high-performance liquid chromatography and mass spectral analysis. J Chromatogr. 1992 Feb. 14; 574(2):283-92 Abstract). Studies suggest that the majority of biological activity is due to the 11-hyroxylated form which has a half-life of approximately four-times that of yohimbine (Le Corre P, Dollo G, Chevanne F, Le Verge R. Biopharmaceutics and metabolism of yohimbine in humans. Eur J Pharm Sci. 1999 October; 9(1):79-84 Abstract).

The main identified biological activity of yohimbine is as an alpha(2) adrenergic blocker or antagonist (Cameron O G, Zubieta J K, Grunhaus L, Minoshima S. Effects of yohimbine on cerebral blood flow, symptoms, and physiological functions in humans. Psychosom Med. 2000 July-August; 62(4):549-59). Blockade of alpha(2) adrenoreceptors by yohimbine has the effect of stimulating the sympathetic nervous system (Le Corre P, Parmer R J, Kailasam M T, Kennedy B P, Skaar T P, Ho H, Leverge R, Smith D W, Ziegler M G, Insel P A, Schork N J, Flockhart D A, O'Connor D T. Human sympathetic activation by alpha2-adrenergic blockade with yohimbine: Bimodal, epistatic influence of cytochrome P450-mediated drug metabolism. Clin Pharmacol Ther. 2004 August; 76(2):139-53), which increases lipid mobilization—an indication of fat breakdown (Millet L, Barbe P, Lafontan M, Berlan M, Galitzky J. Catecholamine effects on lipolysis and blood flow in human abdominal and femoral adipose tissue. J Appl Physiol. 1998 July; 85(1):181-8). This effect is likely due to increased signaling to other adrenoreceptors upon blockage of alpha(2) adrenoreceptors.

A number of studies provide evidence for the efficacy of yohimbine in stimulating increased lipid mobilization. In obese females on a calorie-restricted diet, yohimbine administration increased weight loss from 2.21 kg (placebo) to 3.55 kg (yohimbine) (Kucio C, Jonderko K, Piskorska D. Does yohimbine act as a slimming drug? Isr J Med Sci. 1991 October; 27(10):550-6 Abstract). In another study, oral administration of yohimbine induced lipid mobilization in both obese and non-obese women (Berlan M, Galitzky J, Riviere D, Foureau M, Tran M A, Flores R, Louvet J P, Houin G, Lafontan M. Plasma catecholamine levels and lipid mobilization induced by yohimbine in obese and non-obese women. Int J Obes. 1991 May; 15(5):305-15 Abstract) as well as in healthy men (Galitzky J, Taouis M, Berlan M, Riviere D, Garrigues M, Lafontan M. Alpha 2-antagonist compounds and lipid mobilization: evidence for a lipid mobilizing effect of oral yohimbine in healthy male volunteers. Eur J Clin Invest. 1988 December; 18(6):587-94 Abstract).

Furthermore, yohimbine has been linked to increased plasma levels of leptin, which is known to lead to increased fat breakdown and weight loss (Naghadeh M M, Aghadeh M, Homayouni M F, Ibrahimi H. Effects of yohimbine on plasma levels of leptin in normal and streptozotocin induced diabetic rats. ACTA MEDICA IRANICA 2006; 44:77-82). Leptin is the protein product of the Ob ‘obese’ gene and signals the status of fat storage to the brain to modulate hunger and metabolic rate, likely through regulation of UCP3, an uncoupling protein expressed in a variety of tissue including muscle and brown adipose tissue (Janeckova R. The role of leptin in human physiology and pathophysiology. Physiol Res. 2001; 50(5):443-59).

In an embodiment of the present invention, which is set forth in greater detail in the examples below, the nutritional supplement includes yohimbine or derivatives thereof. A serving of the nutritional supplement may include from about 0.0005 g to about 0.0035 g of yohimbine or derivatives thereof. The preferred dosage of a serving of the nutritional supplement comprises about 0.0020 g of yohimbine or derivatives thereof.

In one embodiment of the present invention, the nutritional supplement includes 11-hydroxyyohimbine as a derivative of yohimbine. The preferred dosage of a serving of the nutritional supplement comprises from about 0.01 mg to about 0.05 mg of 11-hydroxyyohimbine.

Evodia rutaecarpia

Evodia species of plants are a source of many chemicals with a variety of potentially beneficial actions. One specific chemical is evodiamine, which has been shown to increase the secretion of adrenergic signaling molecules and stimulate the sympathetic nervous system (Yoshizumi M, Houchi H, Ishimura Y, Hirose M, Kitagawa T, Tsuchiya K, Minakuchi K, Tamaki T. Effect of evodiamine on catecholamine secretion from bovine adrenal medulla. J Med Invest. 1997 August; 44(1-2):79-82 Abstract). Evodiamine has also been shown to increase energy expenditure and lipid mobilization and decrease body fat in mice (Kobayashi Y, Nakano Y, Kizaki M, Hoshikuma K, Yokoo Y, Kamiya T. Capsaicin-like anti-obese activities of evodiamine from fruits of Evodia rutaecarpa, a vanilloid receptor agonist. Planta Med. 2001 October; 67(7):628-33 Abstract).

In an embodiment of the present invention, which is set forth in greater detail in the examples below, the nutritional supplement includes Evodia rutaecarpia extract. A serving of the nutritional supplement may include from about 0.0003 g to about 0.0050 g of Evodia rutaecarpia extract. The preferred dosage of a serving of the nutritional supplement comprises about 0.0010 g of Evodia rutaecarpia extract.

In one embodiment of the present invention, the nutritional supplement includes Evodia rutaecarpia extract standardized to evodiamine. The preferred dosage of a serving of the nutritional supplement comprises from about 0.05 mg to about 0.50 mg evodiamine.

L-carnitine

L-carnitine is an amino acid available from meat, but can also be synthesized by humans from other amino acids. Studies have shown L-carnitine supplementation is useful for a number of conditions including aiding athletic performance, improving immunity and improving the symptoms of cardiovascular disease and diabetes (Monograph. L-carnitine. Altern Med Rev. 2005 March; 10(1):42-50). The primary mechanism of L-carnitine action is through it's involvement in the transport of fatty acids to the mitochondria for oxidation.

Research has shown that L-carnitine supplementation increases fat oxidation according to a number of measured parameters including a reduction in total cholesterol, LDL and triglycerides (Monograph. L-carnitine. Altern Med Rev. 2005 March; 10(1):42-50). In cultured cancer cells which typically have an altered metabolism to favor cytoplasmic glycolysis over mitochondrial fat oxidation, increasing the oxidation of fat with L-carnitine resulted in a reduction of cancer attributes (Wenzel U, Nickel A, Daniel H. Increased carnitine-dependent fatty acid uptake into mitochondria of human colon cancer cells induces apoptosis. J Nutr. 2005 June; 135(6):1510-4). Increasing total muscle L-carnitine concentration in healthy men decreased carbohydrate oxidation, while offering indications of increased fat oxidation (Stephens F B, Constantin-Teodosiu D, Laithwaite D, Simpson E J, Greenhaff P L. An acute increase in skeletal muscle carnitine content alters fuel metabolism in resting human skeletal muscle. J Clin Endocrinol Metab. 2006 Sep. 19).

Not wishing to be bound by theory, it is believed that the combined action of the constituents of the present invention will act to jointly and simultaneously through complementary yet distinct mechanisms to aid in weight loss or weight maintenance. Yohimbine acts to induce the mobilization of fat and reduce hunger by increasing levels of leptin; Evodia rutaecarpia extract increases thermogenesis while also increasing fat mobilization and L-carnitine enhances the transport of fat to the mitochondria for oxidation.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a powder.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

In another embodiment of the present invention, the composition or portion thereof is provided in the form of fine-milled particles. As used herein, the terms “fine-milled” and/or “fine-milling” refer the process of micronization. Micronization is a mechanical process which involves the application of force to a particle, thereby resulting in a reduction in the size of said particle. U.S. Provisional Application No. 60/776,325 entitled “Compositions and Method for Increasing Bioavailability of Compositions for Performance Improvement”, which is herein fully incorporated by reference discloses a method of improving the absorption, palatability, taste, texture and bioavailability of compounds by increasing the solubility. The increased bioavailability of a compound or ingredients is achieved via a reduction in particle size using a “fine-milling” technique. Any acceptable fine-milling technique wherein the result is the fine-milled particles having an average particle size of between about 50 microns to about 2 microns. The reduction in size of the particles increases the surface area-to-volume ratio of each particle, thus increasing the rate of dissolution, thereby improving the rate of absorption.

The present nutritional composition or those similarly envisioned by one of skill in the art, may be utilized in methods to promote weight loss or maintenance in an individual by increasing thermogenesis and lipid oxidation.

Although the following example illustrates the practice of the present invention in one of its embodiments, the example should not be construed as limiting the scope of the invention. Other embodiments will be apparent to one of skill in the art from consideration of the specifications and example.

### EXAMPLE

A nutritional supplement is provided in one serving per day in powder form. A single serving of the nutritional supplement comprises from about 0.0005 g to about 0.0035 g of yohimbine, from about 0.01 mg to about 0.05 mg of 11-hydroxyyohimbine, from about 0.0003 g to about 0.0050 g of Evodia rutaecarpia extract which has been standardized to contain from about 0.05 mg to about 0.50 mg of evodiamine.

Directions: As a nutritional supplement, at least one serving of the powder is provided daily, up to three servings per day. Said servings are mixed with 8 oz. of water and consumed at least once daily. Each serving may be consumed immediately before exercise.

What is claimed:

1. A composition for promoting weight loss or maintenance in a mammal comprising:
a source of an effective amount of yohimbine or derivatives thereof, an effective amount of Evodia rutaecarpia extract and a source of an effective amount of L-carnitine or derivatives thereof,
whereby said composition acts to jointly and simultaneously support, facilitate and otherwise increases lipid oxidation and thermogenesis.

2. A method for promoting weight loss or maintenance in a mammal comprising:
administering an effective amount of yohimbine or derivatives thereof, an effective amount of Evodia rutaecarpia extract and a source of an effective amount of L-carnitine or derivatives thereof to said mammal,
whereby said composition acts to jointly and simultaneously support, facilitate and otherwise increases lipid oxidation and thermogenesis.

3. The composition of claim 1 further comprising at least one of the following:
Yerba Mate, Black tea, Theobroma cacao, White tea dry leaf extract, Caffeine Anhydrous and White willow Bark.

4. The method of claim 2 further comprising at least one of the following:
Yerba Mate, Black tea, Theobroma cacao, White tea dry leaf extract, Caffeine Anhydrous and White willow Bark.
